(19) United States
(12) Patent Application Publication
(10) Pub. No.: US 2026/0146634 A1
(43) Pub. Date: May 28, 2026

(54) APPARATUS FOR MECHANICALLY CONNECTING A COMPONENT TO A GANTRY

(71) Applicant: Siemens Healthineers AG, Forchheim (DE)

(72) Inventors: Patrick LUDWIG, Erlangen (DE); Dominik TRAUTNER, Forchheim (DE); Andreas HADER, Erlangen (DE)

(73) Assignee: Siemens Healthineers AG, Forchheim (DE)

(21) Appl. No.: 19/402,349

(22) Filed: Nov. 26, 2025

(30) Foreign Application Priority Data
Nov. 27, 2024 (DE) 10 2024 211 325.5

(51) Int. Cl. F16B 5/02; A61B 6/00; F16B 31/04

(52) U.S. Cl. CPC F16B 5/0241; A61B 6/44; F16B 31/04

(57) ABSTRACT

An apparatus for mechanically connecting a component to a gantry, comprises: a fastener fitted through a through-opening in the component, wherein the fastener is screwed to the gantry; and a spring assembly positioned on the fastener. The spring assembly includes one or more springs, wherein the one or more springs are arranged between a head of the fastener and the component. The one or more springs are compressed to generate a pretensioning that securely holds the component on the gantry. The component has a fastening portion in which the through-opening is arranged, wherein the through-opening has a tapered widening on a side facing the gantry. The fastener has a positive connecting portion, wherein the positive connecting portion is positively arranged in the tapered widening.

### CROSS-REFERENCE TO RELATED APPLICATION(S)

The present application claims priority under 35 U.S.C. § 119 to German Patent Application No. 10 2024 211 325.5, filed Nov. 27, 2024, the entire contents of which are incorporated herein by reference.

### FIELD

One or more example embodiments of the present invention relate to an apparatus for mechanically connecting a component to a gantry, comprising a fastener (or fastening means) which is guided through a through-opening in the component, a gantry to which the fastener is screwed and a spring assembly which is positioned on the fastener, wherein the spring assembly comprises one or more springs, wherein the at least one spring is arranged between a head of the fastener and the component, wherein the at least one spring is compressed in a state of the fastener screwed to the gantry, in order to generate a pretensioning which securely holds the component on the gantry.

One or more example embodiments of the present invention relate to the technical field of medical technology, in particular the mechanical connection of components to a gantry in computed tomograph devices. The specific subject area comprises the secure fastening of components, taking into account the high mechanical stresses and thermal expansion which are produced by the rotation of the gantry. One or more example embodiments of the present invention are particularly relevant for monitoring and stabilizing components in rotating medical appliances, in order to ensure the accuracy and reliability of the diagnostic methods.

### BACKGROUND

Significant radial forces occur during the rotation of a computed tomography device, which lead to deformations of the rotating drum/disk. These deformations are transferred to the components installed in the gantry and generate high mechanical stresses therein. Previous solutions comprise the direct screwing of the components or the use of spring assemblies in order to compensate for the thermal expansion. However, these methods do not ensure the complete decoupling of the mechanical stresses which are produced by the deformation of the drum/disk.

Current developments in the field of medical technology are concentrated on the improvement of the mechanical stability and the reduction of stresses in the components. For example, different approaches for damping and decoupling mechanical forces are described in the literature. In spite of these advances there are still challenges in the effective decoupling of the components from the stresses caused by the rotation.

### SUMMARY

A technical problem which will be solved by one or more example embodiments of the present invention is not to transfer to the fastened components the mechanical stresses which are produced by the deformation of the drum/disk in a computed tomography device. An object of one or more example embodiments of the present invention, in particular, is to provide an apparatus which permits a secure and stress-free fastening of the components to the gantry and, in particular, is simple and cost-effective in production and/or use.

One or more example embodiments of the present invention solves at least the problem defined above by providing an apparatus for mechanically connecting a component to a gantry, which comprises a fastener, a through-opening in the component, a gantry and a spring assembly. The spring assembly, which contains one or more springs, is arranged between the head of the fastener and the component and compressed in a screwed state in order to generate a pretensioning. This pretensioning securely holds the component on the gantry and prevents the transfer of mechanical stresses to the component.

Independent of the grammatical term usage, individuals with male, female or other gender identities are included within the term.

In a first aspect, one or more example embodiments of the present invention relate to an apparatus for mechanically connecting a component to a gantry, comprising:
a fastener which is guided through a through-opening in the component,
a gantry to which the fastener is screwed,
a spring assembly which is positioned on the fastener,
   wherein the spring assembly comprises one or more springs,
   wherein the at least one spring is arranged between a head of the fastener and the component, wherein the at least one spring is compressed in a state of the fastener screwed to the gantry, in order to generate a pretensioning which securely holds the component on the gantry,
   wherein the component has a fastening portion in which the through-opening is arranged, wherein the through-opening has a tapered widening on the side facing the gantry, wherein the fastener has a positive connecting portion, wherein the positive connecting portion is positively arranged in the tapered widening.

The apparatus comprises a fastener which is guided through a through-opening in the component. The fastener serves to connect the component mechanically to the gantry. A fastener is an element which is used in order to connect together two or more parts. Common examples are screws, bolts or rivets. A through-opening is an opening which passes entirely through a material so that the fastener can be guided through. The fastener can take different forms, such as for example a screw, a bolt or a pin. The through-opening is substantially cylindrical but can have a tapered portion, for example in one or both end regions. Preferably, the fastener forms a screw which is guided through a hole as a through-opening, in order to fasten it to a gantry. The fastener can be based on and/or consist of different materials, such as for example steel, titanium or plastics, depending on the specific requirements of use.

The apparatus comprises a gantry to which the fastener is screwed. A gantry is a structure which serves as a frame or carrier and can be fastened to the other components. The gantry is preferably a part of a computed tomography device to which the component is fastened. The gantry forms the structure which carries and stabilizes the rotating parts of the appliance. The gantry can consist of different materials such as aluminum, steel or composite materials and have different designs in order to meet the specific requirements of the computed tomography device. A typical example of a gantry is the rotating structure of a computed tomography device which carries the X-ray source and the detectors. The gantry serves as a stable base for the fastening of the component and contributes to the overall stability and functionality of the computed tomography device.

Typical rotational speeds of a gantry in a computed tomography device are between 1 and 4 revolutions per second which leads to significant centrifugal forces which act on the components in the order of several g. These forces set particular requirements for the fastening of a component and the fastener.

The apparatus comprises a spring assembly which is positioned on the fastener, wherein the spring assembly comprises one or more springs. A spring assembly is an arrangement of one or more springs which cooperate in order to fulfill a specific function. A spring is a resilient element which can store and release mechanical energy. The spring assembly can comprise different types of springs, such as for example compression springs, tension springs or torsion springs. The number and type of springs can vary depending on the requirements of use. The spring assembly, in particular, ensures a resilient connection between the fastener and the component, whereby a pretensioning is generated which securely holds the component on the gantry. The apparatus, in particular the spring assembly, comprises at least one spring which is arranged between a head of the fastener and the component, wherein the at least one spring is compressed in a state of the fastener screwed to the gantry, in order to generate a pretensioning which securely holds the component on the gantry. A pretensioning is a force which is generated in a spring when it is compressed or expanded in order to maintain a specific tension. The head of the fastener is the part of the fastener which normally has a larger diameter and serves to hold the fastener in situ. The pretensioning can be generated by different types of springs and the strength of the pretensioning can be adapted depending on the requirements of use. One example might be a screw with a compression spring which is compressed when the screw is tightened in order to maintain a constant tension. The pretensioning ensures that the component is securely held on the gantry by exerting a constant force on the connection.

The apparatus comprises a component which has a fastening portion in which the through-opening is arranged. This component is designed to be fastened to the gantry and is subjected to radial forces during operation. The fastening portion is the part of the component which is specifically designed to receive and to hold the fastener securely. This portion can be reinforced or differently designed in order to meet the mechanical requirements. The fastening portion is preferably a base portion or a frame. The fastening portion can be, for example, a metal base plate or a base plate.

The through-opening which is located in this fastening portion has a tapered widening on the side facing the gantry. This tapered winding is a widening of the opening which has a tapered shape in order to ensure an improved fit and stability of the fastener. When inserted, the fastener is centered and stabilized by the tapered shape, which leads to a stronger and more secure connection.

The fastener itself has a positive connecting portion which is arranged at least partially positively in the tapered widening. A positive connecting portion is a specially shaped part of the fastener which is designed such that it is inserted positively in the tapered widening. This means that the positive connecting portion and the tapered widening are adapted to one another such that they form a strong and stable connection which withstands mechanical loads. Preferably, the fastener has a threaded portion and this is arranged, in particular, at one end opposing the head of the fastener. The positive connecting portion preferably adjoins the threaded portion and/or is arranged between a center of the fastener and the end comprising the thread. The positive connecting position, in particular, is configured continuously circumferentially. In particular, the positive connecting portion can have circumferential recesses.

One or more example embodiments of the present invention permit a connection of the component and gantry which is secure and can be implemented cost-effectively in terms of technology and which withstands the high rotational forces which occur during operation. This construction ensures that the mechanical connection between the component and the gantry itself remains reliable even under extreme conditions. In particular, the positive connection between the positive connecting portion of the fastener and the tapered widening of the through-opening leads to a fixed bearing which securely fixes the component. At the same time, this arrangement permits the compensation of stresses which can arise due to the dynamic loads during rotation.

Particularly preferably, the through-opening forms a through-bore. This means that the opening passes entirely through the component. A through-bore permits a simple and accurate mounting of the fastener since the fastener is guided directly through the component and can be screwed to the gantry. An example thereof is a bore in a metal plate through which a screw is guided in order to fasten the plate to a structure. This type of bore ensures that the fastener sits firmly and securely, which is particularly important if the connection is subjected to high loads.

In particular, the tapered widening of the through-opening is conical, semi-spherical or spherical portion-shaped. This shaping of the widening permits an improved distribution of the forces which act on the connection and thus increases the stability of the connection. A conical or spherical portion ensures that the fastener is located positively in the widening which additionally stabilizes the connection. An example thereof is a conical recess in a metal plate into which the positive connecting portion of tapered shape fits. This shaping prevents the fastener from being released or displaced under load and ensures a uniform distribution of the forces along the connection.

The tapered widening is preferably conically configured, wherein the positive connecting portion of the fastener forms a spherical portion or is configured to be semi-spherical. This combination permits a flexible and yet stable connection which can be adapted to different angles and positions. A spherical portion on the fastener fits into the conical widening of the through-opening and thus permits a certain mobility of the connection without impairing the stability.

The apparatus preferably comprises at least one additional fastener which is guided through an additional through-opening in the component and is screwed to the gantry. The additional fastener has an additional spring assembly which is positioned on the additional fastener. The additional spring assembly comprises one or more springs which are arranged between a head of the additional fastener and the component. In a state of the additional fastener screwed to the gantry, the at least one spring is compressed in order to generate a pretensioning which holds the component on the gantry. The additional fastener forms, in particular, a floating bearing for the fastening of the component to the gantry.

Optionally the apparatus comprises at least two additional fastener, wherein the through-opening for the fastener is arranged between two additional through-openings. This arrangement permits a uniform distribution of the forces and increases the stability of the connection. In this case the connections of the component via the additional fastener form a floating bearing and the connection arranged therebetween via the fastener forms a fixed bearing. The floating bearings substantially serve for the general connection of the component and gantry, wherein the fastener as a fixed bearing further stabilizes this connection and at the same time permits a deformation in a secure manner during operation.

A fixed bearing fixes the component in a fixed position, while a floating bearing permits a certain mobility in order to compensate for thermal expansion or other movements.

The fastening portion of the component has, in particular, a lower stiffness than the gantry. This means that the fastening portion is more flexible and can be adapted more easily to the shape and position of the gantry. An example thereof is a component made of a flexible material which can be easily deformed in order to ensure a perfect fit with the gantry, while the gantry itself consists of a stiffer material which provides the required stability.

The fastener and/or the additional fastener are configured, in particular, in a pin-shaped manner. A pin-shaped configuration permits a simple and accurate mounting, since the pins can be easily guided through the bores in the component and screwed to the gantry.

A further aspect of one or more example embodiments of the present invention relates to the use of the apparatus. The apparatus is used for the secure fastening of a component to a gantry in a medical apparatus. The fastener is guided through a through-opening in the component and is screwed to the gantry. A spring assembly which comprises one or more springs is positioned on the fastener in order to generate a pretensioning which holds the component securely on the gantry. An example thereof is the fastening of an X-ray detector to a gantry in a medical imaging appliance. The spring assembly ensures that the detector is held fixedly and securely on the gantry even in the case of vibrations or movements of the appliance.

The apparatus is used for the secure fastening of a component to a gantry in a medical apparatus, wherein in particular at least one additional fastener is guided through an additional through-opening in the component and is screwed to the gantry. An additional spring assembly which comprises one or more springs is positioned on the additional fastener in order to generate a pretensioning which holds the component on the gantry.

A computed tomography device forms a further aspect of one or more example embodiments of the present invention, wherein the computed tomography device comprises a gantry and a component fastened thereto according to the described apparatus and use. The fastener and the spring assembly securely hold the component on the gantry. An example thereof is a computed tomography device in which the X-ray detector is securely fastened to the gantry, in order to ensure an accurate imaging. The spring assembly ensures that the detector is fixedly held on the gantry even during the rapid movements and vibrations which can occur during the scanning process.

These features and examples illustrate the wide variety of possible uses of the apparatus for the secure fastening of components to a gantry in medical apparatuses. The use of spring assemblies and additional fastener ensures a stable and secure connection which meets the requirements in medical environments.

### BRIEF DESCRIPTION OF THE DRAWINGS

Further advantages, effects and embodiments are found in the accompanying figures and the description thereof, in which:

FIG. 1 shows a schematic view of a computed tomography device according to the prior art,

FIG. 2 shows a schematic view of a gantry with a gantry frame with supporting arms according to the prior art,

FIG. 3 shows an apparatus for mechanically connecting a component to a gantry,

FIG. 4a and FIG. 4b show a detailed view and sectional image of an apparatus for mechanically connecting a component to a gantry, and

FIG. 5a and FIG. 5b show a fastener and an additional fastener of an apparatus according to FIG. 4a, b.

### DETAILED DESCRIPTION

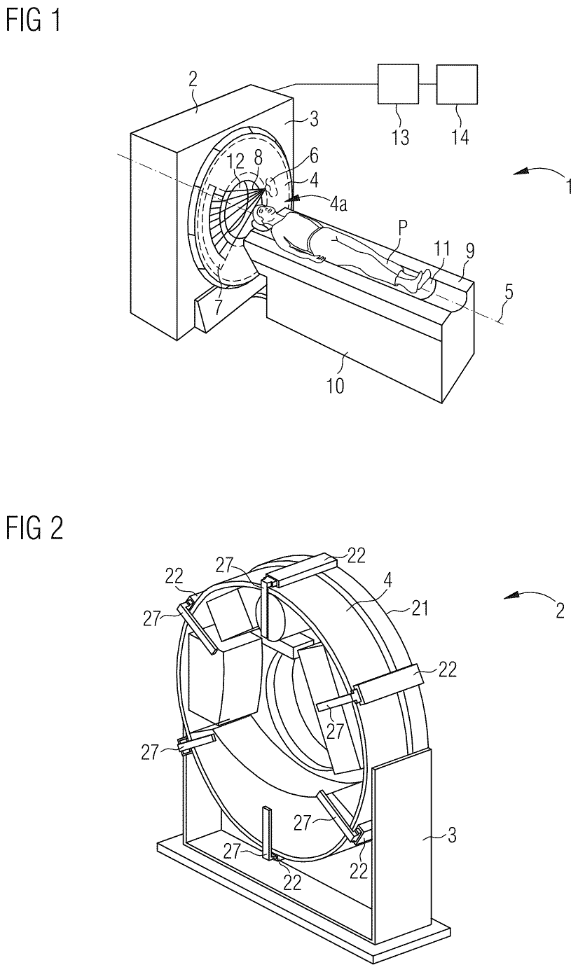

FIG. 1 shows a general schematic view of a computed tomography device 1 in order to illustrate the general construction thereof. The arrangement comprises a gantry 2 with a stationary part 3, also denoted as the gantry frame, and with a part 4a which is rotatable or rotating about a system axis 5, with a drum 4. The rotating part 4a which has the function of an imaging system (X-ray system) 4a also comprises, in addition to the drum, an X-ray source 6 and an X-ray detector 7 which are opposingly arranged in the drum 4. The X-ray source 6 and X-ray detector 7 form together with the drum 4 the imaging system 4a. During the operation of the computed tomography device 1, X-ray radiation 8 is emitted from the X-ray source 6 in the direction of the X-ray detector 7, it penetrates a measurement object P, for example a patient P, and is detected by the X-ray detector 7 in the form of measurement data or measurement signals.

A patient couch 9 for supporting a patient P can also be seen in FIG. 1. The patient couch 9 comprises a couch base 10 on which a patient support table 11 provided for the actual support of the patient P is arranged. The patient support table 11 is adjustable relative to the couch base 10 in the direction of the system axis 5 such that it can be introduced together with the patient P into an opening 12 or the patient receiving region 12 of the gantry 2 for recording X-ray projections of the patient P. The computerized processing of the X-ray projections recorded by the imaging system 4a or the reconstruction of tomographic images, 3D images or a 3D data set based on the measurement data or measurement signals of the X-ray projections is carried out by an image processor 13 of the computed tomography device 1, wherein the tomographic images or 3D images can be displayed on a display apparatus 14. The image processor 13 can also be configured as a control facility for a control of an imaging process for activating the gantry 2 and, in particular, the imaging system 4a.

So that the rotating part 4a forming the imaging system 4a can rotate relative to the stationary part 3 of the gantry 2, a support of the rotating part 4a is required. To this end, for example, an annular rolling bearing is used, which annular rolling bearing is axially arranged approximately in the center of the gantry 2 and circulates in an annular manner around the patient receiving region 12.

A gantry 2 with a conventional gantry frame 3 with supporting arms 22 is shown in FIG. 2. The gantry frame 3 comprises a supporting carrier structure 21 on the rear face of the gantry frame 3. The carrier structure 21 is configured to be relatively narrow in the axial direction but in the radial direction it has a large diameter for receiving a cylindrical drum 4. A plurality of supporting arms 22 which run in the axial direction and protrude over the drum 4 are arranged on the outer faces of the carrier structure 21. So-called holders 27 which run at an angle of 90° to the supporting arms 22 or radially to the axis of rotation of the drum 4 are fastened to the supporting arms 22. Functional elements, such as for example a control panel, a display and similar units (not shown) which are designed to be accessible from the front face of the gantry 2, can be fastened to the holders 27.

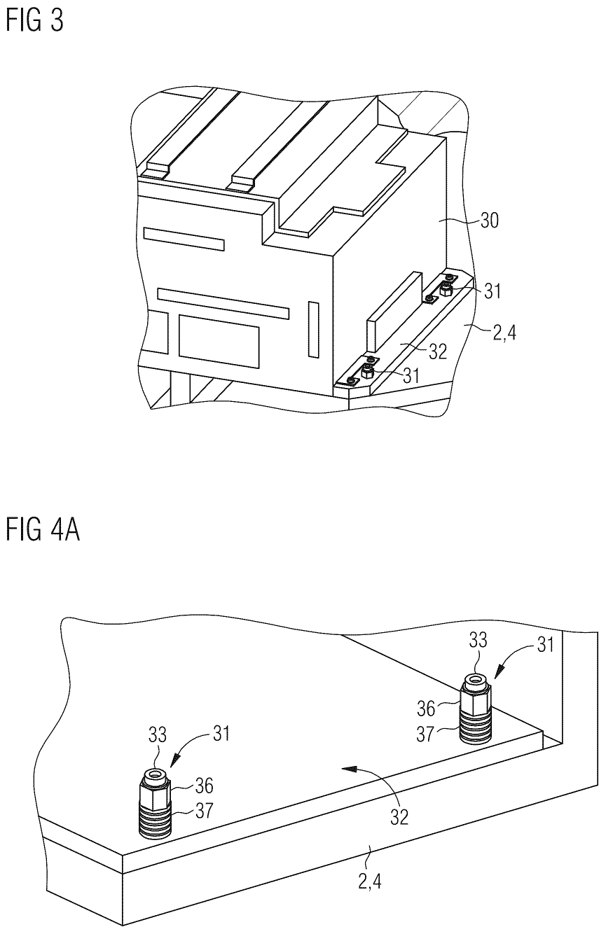

FIG. 3 shows a detail of a computed tomography device 1. The detail shows once again a detail of the gantry 2 and a component 30 fastened thereto. The component 30 is attached via a fastener 31 to the gantry 2, in particular to the drum 4. The component 30 is, for example, an electronic component. The component 30 has a fastening portion 32 which is configured here as a projection or flange. Alternatively and/or additionally, the fastening portion 32 can form a base plate or a portion of a base plate. When the drum 4 is rotated, the component 30 rotates therewith, so that during the operation of the computed tomography device 1 the component 30 is subjected to high forces, in particular radial forces and/or centrifugal forces. When at a standstill, the component 30 is subjected to gravity, which has different effects depending on the position of the component (for example 9 o'clock or 12 o'clock). The attachment and/or fastening of the component 30 to the gantry 2 or to the drum 4 has to be designed such that it withstands these forces and it is possible to compensate for any resulting deformations. This is provided by the fastening according to one or more example embodiments of the present invention.

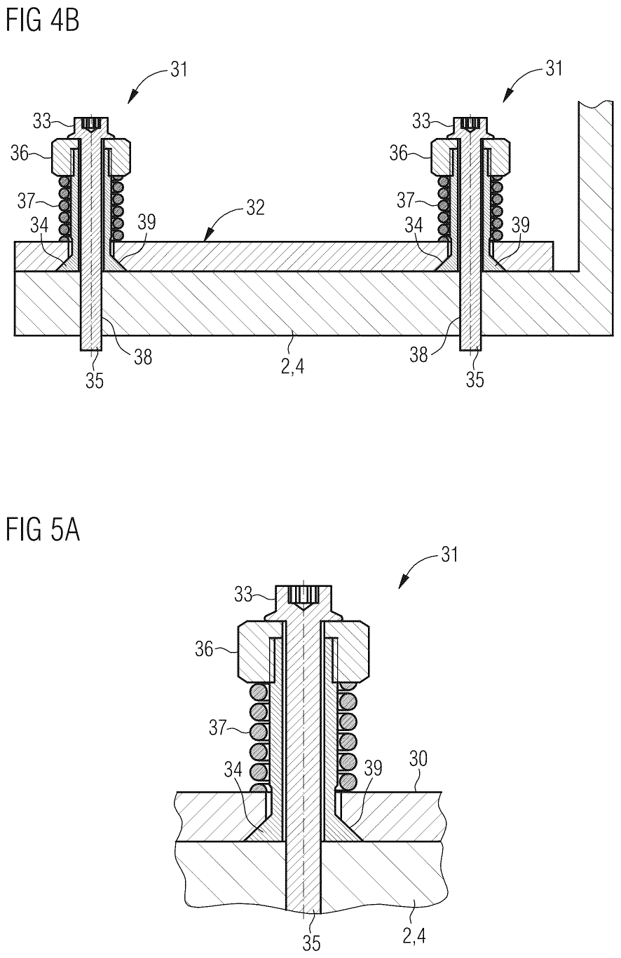

FIGS. 4a and 4b show a simplified detail of a fastening of a component 30 to the gantry 2 of a computed tomography device 1. For example, in this case it can be the fastening of the component 30 of FIG. 3, wherein for simplification only the base plate and/or the fastening portion 32 are shown and all further details such as the electronic component itself are omitted. The fastening portion 32 is preferably arranged in an edge region and/or rim region of the component 30 or the base plate. Two fasteners 31 which are arranged at the ends in the fastening portion 32 are shown here.

The fastener 31 has a head 33 which preferably is configured, in cooperation with a corresponding tool, for example a screwdriver, to screw the fastener 31 to the gantry two and/or the drum four. Below the head 33 the fastener 31 is configured substantially cylindrically, wherein “substantially” means in this case excluding the positive connecting portion 34.

The fastening portion 32 has two through-openings 35. The through-openings 35 are based substantially on a through-bore in the base plate and/or the component 30. The fastener 31 is guided through the through-opening 35. A nut 36 which is in engagement, in particular, with the thread of the fastener 31 is arranged in the region of the head 33 of the fastener 31.

A spring assembly 37 is positioned on the fastener 31 between the nut 36 and the fastening portion 32. The spring assembly 37 comprises at least one spring and can be adjusted and/or regulated in terms of pretensioning by and/or via the nut 36. The pretensioning is adjusted such that the spring assembly 37 and/or the springs push the component 30 against the gantry 2 and/or the drum four.

At the opposing end of the head 33 the fastener 31 has a thread 38. The fastener 31 is screwed via the thread 38 to the gantry 2 and/or the drum 4. In other words, the fastener 31 and the component 30 are held on the gantry 2 and/or the drum 4 by this screw connection.

The fastener 31 has a positive connecting portion 34. The positive connecting portion is arranged in a central third along the longitudinal extent of the fastener 31. The positive connecting portion 34 forms, for example, a cone portion and/or spherical circumferential collar and/or protrusion.

Preferably, the positive connecting portion 34 is configured from the same material, for example metal or a metal alloy, as the fastener 31; alternatively the positive connecting portion 34 can be constructed and/or formed from a different material, in particular positioned on the cylindrical portion of the fastener 31.

The component 30, in particular the through-opening 35, has a tapered widening 39 on the side facing the gantry 4. The tapered widening 39 is preferably configured to be conical, for example a conical bore of the through-opening 35. In a fastened state of the component 30, the positive connecting portion 34 is arranged in the tapered widening 39. In particular, it is provided that the positive connecting portion 34 forms a spherical portion and the tapered widening 39 forms a conical widening. The positive connecting portion 34 is arranged by a positive connection, in particular a non-positive and/or frictional connection, in the tapered widening 39. Due to the positive connection of the positive connecting portion 34 and the tapered widening 39 the fastener 31 forms a fixed bearing point.

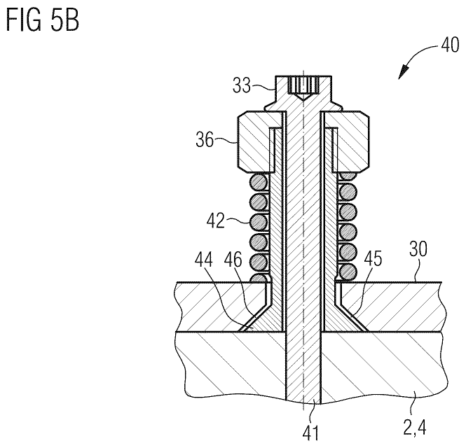

FIG. 5a shows the fastener 31 of FIG. 4a and 4b, wherein it can be identified here that the positive connecting portion 34 is positively arranged in the tapered widening 39, so that the fastener 31 forms a fixed bearing.

FIG. 5b shows an additional fastener 40 which is substantially constructed in the manner of the fastener 31. The additional fastener 40 is guided through an additional through-opening 41 in the component 30 and screwed to the gantry 2. The additional fastener 40 comprises an additional spring assembly 42 which is positioned on the additional fastener 40, wherein the additional spring assembly 42 comprises one or more springs, wherein the at least one spring is arranged between a head 33 of the additional fastener 40 and the component 30, wherein the at least one spring is compressed in order to generate a pretensioning which holds the component 30 on the gantry 2.

In contrast to the fastener 31, the additional fastener can be configured without a positive connecting portion 34 or can have a smaller additional positive connecting portion 44. The additional positive connecting portion 44 is configured to be smaller than a tapered additional widening 45 and thus is not in positive engagement therewith. A gap 46 is arranged between the additional positive connecting portion 44 and the tapered additional widening 45. This can be generated, for example, by the tapered additional widening 45 being widened further than the tapered widening 39 so that the additional positive connecting portion 44 is too small to be held positively. Alternatively and/or additionally, the additional positive connecting portion 44 can be configured to be smaller relative to the positive connecting portion 34, for example by material having been removed from a positive connecting portion 34. Such an additional fastener 40 forms a floating bearing point.

According to one embodiment, it is preferably provided that in each case a fastener 31 is arranged between two additional fasteners 40. Due to its design as a fixed bearing, the fastener 31 forms an additional stabilizing and fixing, in particular for states in which the component 30 attached thereto comes to a standstill at 3 o'clock or 9 o'clock after the rotation of the gantry 2.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. An apparatus for mechanically connecting a component to a gantry, the apparatus comprising:
a fastener fitted through a through-opening in the component, wherein the fastener is screwed to the gantry; and
a spring assembly positioned on the fastener, the spring assembly including one or more springs, wherein the one or more springs are arranged between a head of the fastener and the component, and wherein the one or more springs are compressed to generate a pretensioning that holds the component on the gantry; wherein
the component has a fastening portion in which the through-opening is arranged,
the through-opening has a tapered widening on a side facing the gantry,
the fastener has a positive connecting portion, and
the positive connecting portion is positively arranged in the tapered widening.

2. The apparatus as claimed in claim 1, wherein the through-opening forms a through-bore.

3. The apparatus as claimed in claim 1, wherein the tapered widening is conical or semi-spherical.

4. The apparatus as claimed in claim 1, wherein the tapered widening is conical and the positive connecting portion forms a spherical portion.

5. The apparatus as claimed in claim 1, further comprising:
at least one additional fastener fitted through an additional through-opening in the component and screwed to the gantry, wherein
the at least one additional fastener has an additional spring assembly positioned on the at least one additional fastener,
the additional spring assembly includes one or more springs,
the one or more springs are arranged between a head of the at least one additional fastener and the component, and
the one or more springs are compressed to generate a pretensioning that holds the component on the gantry.

6. The apparatus as claimed in claim 5, further comprising:
at least two additional fasteners, wherein
the through-opening for the fastener is arranged between two additional through-openings.

7. The apparatus as claimed in claim 5, wherein the fastener forms a fixed bearing and the at least one additional fastener forms a floating bearing.

8. The apparatus as claimed in claim 1, wherein the fastening portion has a lower stiffness than at least one of the gantry or a drum.

9. The apparatus as claimed in claim 5, wherein at least one of the fastener or the at least one additional fastener is pin-shaped.

10. A method for use of the apparatus as claimed in claim 1, the method comprising:
securely fastening the component to the gantry by guiding the fastener through the through-opening in the component and screwing the fastener to the gantry.

11. The method as claimed in claim 10, wherein
at least one additional fastener is guided through an additional through-opening in the component and screwed to the gantry, and
an additional spring assembly, which includes one or more springs, is positioned on the at least one additional fastener to generate a pretensioning that holds the component on the gantry.

12. A computed tomography device, comprising:
a gantry; and
a component fastened to the gantry via the apparatus as claimed in claim 1.

13. The apparatus as claimed in claim 2, wherein the tapered widening is conical or semi-spherical.

14. The apparatus as claimed in claim 13, wherein the tapered widening is conical and the positive connecting portion forms a spherical portion.

15. The apparatus as claimed in claim 14, further comprising:
at least one additional fastener fitted through an additional through-opening in the component and screwed to the gantry, wherein
the at least one additional fastener has an additional spring assembly positioned on the at least one additional fastener,
the additional spring assembly includes one or more springs,
the one or more springs are arranged between a head of the at least one additional fastener and the component, and
the one or more springs are compressed to generate a pretensioning that holds the component on the gantry.

16. The apparatus as claimed in claim 15, further comprising:
at least two additional fasteners, wherein
the through-opening for the fastener is arranged between two additional through-openings.

17. The apparatus as claimed in claim 14, wherein the fastener is pin-shaped.

18. The apparatus as claimed in claim 13, further comprising:
at least one additional fastener fitted through an additional through-opening in the component and screwed to the gantry, wherein
the at least one additional fastener has an additional spring assembly positioned on the at least one additional fastener,
the additional spring assembly includes one or more springs,
the one or more springs are arranged between a head of the at least one additional fastener and the component, and
the one or more springs are compressed to generate a pretensioning that holds the component on the gantry.

19. The apparatus as claimed in claim 2, wherein the tapered widening is conical and the positive connecting portion forms a spherical portion.

20. The apparatus as claimed in claim 6, wherein the fastener forms a fixed bearing and the at least one additional fastener forms a floating bearing.
